(19) United States
(12) Patent Application Publication
(10) Pub. No.: US 2025/0383335 A1
(43) Pub. Date: Dec. 18, 2025

(54) Methods and Systems for Measurement, Reporting, Validation, and Optimization of an Amount of Carbon Dioxide Captured and Stored in Liquid or Water

(71) Applicant: Vycarb Inc., Brooklyn, NY (US)

(72) Inventors: Frank Garrett Boudinot, Brooklyn, NY (US); Benjamin P. Smith, Brooklyn, NY (US); Lydia Keppler, Brooklyn, NY (US); Lindsey Dukles, Brooklyn, NY (US)

(21) Appl. No.: 19/236,371

(22) Filed: Jun. 12, 2025

Related U.S. Application Data
(60) Provisional application No. 63/659,188, filed on Jun. 12, 2024.

(51) Int. Cl. G01N 33/18

(52) U.S. Cl. CPC G01N 33/18

(57) ABSTRACT

An example method for quantifying an amount of carbon dioxide captured from liquid is described including receiving, from an autonomous measurement sensing system, a measured amount of dissolved aqueous carbon, which is generated by dosing of material into a container holding the liquid, and a measured amount of carbon dioxide reduction (CDR) in the liquid held in the container, and performing a verification of a volume of carbon captured and stored as bicarbonate and carbonate based on a comparison of the measured amount of dissolved aqueous carbon and the measured amount of CDR. Additional verifications can be performed based on a comparison of the measured amount of CDR and a calculated amount of CDR. Following, based on either of the first verification or the second verification failing, the method includes providing instructions to change an amount of the dosing of the material into the container.

### CROSS REFERENCE TO RELATED APPLICATION

The present disclosure claims priority to U.S. provisional application No. 63/659,188, filed on Jun. 12, 2024, the entire contents of which are incorporated herein by reference.

### FIELD

This disclosure relates generally to the field of water treatment, and more specifically, to new and useful systems and methods for capturing carbon from liquid/water and verifying a quantification of an amount of carbon captured.

### BACKGROUND

The increase in atmospheric greenhouse gas concentrations is a critical issue that has far-reaching environmental and social consequences. The need to reduce these gas concentrations has led to the development of a range of carbon dioxide removal and storage technologies, including the pumping of supercritical carbon dioxide into rock formations for mineral precipitation. While these approaches hold significant promise, scaling them up to achieve meaningful reductions in atmospheric greenhouse gases presents significant challenges. These challenges include the verification and quantification of the mass or volume of carbon dioxide removed, captured, and stored, giving difficulties of carbon sensing and measurement; the cost- effectiveness of the capture and storage of carbon dioxide, given the infrastructure, energy, and technology costs; and the scalability of the solutions, given their dependence on specific inputs and environmental conditions like geologic formations suitable for storage. Additionally, there are challenges in making carbon capture and sequestration solutions that can be low cost, accountable, and easily scaled.

Thus, there is a need to create a new and useful system and method for environmental management of carbon dioxide that can leverage lower-cost, scalable carbon sinks (e.g., in liquid or water systems). There is also a need to quantify and verify an amount of carbon dioxide that is removed, captured, and stored. Examples herein provide such new and useful systems and methods.

### SUMMARY

In one example, a method for quantifying an amount of carbon dioxide captured from liquid is described. The method includes receiving, from an autonomous measurement sensing system, information indicating a measured amount of dissolved aqueous carbon (e.g., such as carbon ions, including carbon dioxide, bicarbonate, and carbonate ions), which is generated by dosing of material into a container holding the liquid that reacts with carbon dioxide in the liquid (e.g., which is put into the liquid either from natural or intentional concentration). The method also includes receiving, from the autonomous measurement sensing system, information indicating a measured amount of carbon dioxide reduction (CDR) in the liquid held in the container, which is calculated by measurements of the liquid at an input to the container pre-treatment and the liquid at an output of the container post-treatment after dosing of the material into the container for reaction with the carbon dioxide in the liquid (e.g., generating the bicarbonate and/or carbonate ions as a form of CDR). The method also includes performing, by a control system including a processor executing first instructions, a first verification of a volume of carbon captured and stored as bicarbonate and carbonate in the container based on a comparison of the measured amount of dissolved aqueous carbon and the measured amount of carbon dioxide reduction (CDR). The method also includes receiving a measurement of alkalinity of the liquid held in the container after the dosing of material into the container, based on the measurement of alkalinity of the liquid and the measured amount of dissolved aqueous carbon, generating a calculated amount of carbon dioxide reduction (CDR), and performing, by the control system including the processor executing second instructions, a second verification of the volume of carbon captured and stored as bicarbonate and carbonate based on a comparison of the measured amount of carbon dioxide reduction (CDR) and the calculated amount carbon dioxide reduction (CDR). The method further includes based on either of the first verification or the second verification failing, providing instructions to change an amount of the dosing of the material into the container.

In another example, a system for quantifying an amount of carbon dioxide captured from liquid is described. The system includes a container including an inlet to receive liquid and an outlet to release the liquid, the container for holding the liquid, an autonomous measurement sensing system including one or more sensors coupled to the container to measure carbon content in the liquid that is held in the container, and a control system including a processor for executing instructions to perform functions. The functions include receiving, from the autonomous measurement sensing system, information indicating a measured amount of dissolved aqueous carbon, which is generated by dosing of material into the container holding the liquid that reacts with carbon dioxide in the liquid. The functions also include receiving, from the autonomous measurement sensing system, information indicating a measured amount of carbon dioxide reduction (CDR) in the liquid held in the container, which is calculated by measurements of the liquid at an input to the container pre-treatment and the liquid at an output of the container post-treatment after dosing of the material into the container for reaction with the carbon dioxide in the liquid. The functions also include performing a first verification of a volume of carbon captured and stored as bicarbonate and carbonate in the container based on a comparison of the measured amount of dissolved aqueous carbon and the measured amount of carbon dioxide reduction (CDR). The functions also include receiving a measurement of alkalinity of the liquid held in the container after the dosing of material into the container, based on the measurement of alkalinity of the liquid and the measured amount of dissolved aqueous carbon, generating a calculated amount of carbon dioxide reduction (CDR), and performing a second verification of the volume of carbon captured and stored as bicarbonate and carbonate based on a comparison of the measured amount of carbon dioxide reduction (CDR) and the calculated amount carbon dioxide reduction (CDR). The functions also include based on either of the first verification or the second verification failing, providing instructions to change an amount of the dosing of the material into the container.

Within examples, carbon is described as captured. In other examples, carbon is captured and stored. Still in other examples carbon is captured and removed, or captured, stored, and removed. Any combination of captured, stored, removed, is included for processing of carbon within examples herein.

The features, functions, and advantages that have been discussed can be achieved independently in various examples or may be combined in yet other examples. Further details of the examples can be seen with reference to the following description and drawings.

### BRIEF DESCRIPTION OF DRAWINGS

Examples, objectives and descriptions of the present disclosure will be readily understood by reference to the following detailed description of illustrative examples when read in conjunction with the accompanying drawings, wherein:

FIG. 1 illustrates a workflow diagram of an example of a system for processing liquid to change an amount of carbon dioxide in the liquid, according to an example implementation.

FIG. 2 illustrates a workflow diagram of another example of the system for processing liquid to change an amount of carbon dioxide in the liquid, according to an example implementation.

FIG. 3 illustrates an example of the system in use, according to an example implementation.

FIGS. 4A-4C are a set of schematic representations of different system variations for optimizing introduction of alkalinity and carbon for improved dissolution and carbon capture kinetics.

FIG. 5 is a block diagram illustrating inputs and outputs applicable to any of the systems illustrated in FIGS. 1-4, according to example implementations.

FIG. 6 is another block diagram illustrating the overall processing of water to generate CDR as performed by any of the systems illustrated in FIGS. 1-4, according to example implementations.

FIG. 7 shows a flowchart of an example of a method for quantifying an amount of carbon dioxide captured from liquid, according to an example implementation.

FIG. 8 is a graph illustrating dissolved alkalinity versus dissolved inorganic carbon (DIC) (referred to herein as aqueous carbon), according to an example implementation.

FIG. 9 is a vector slope diagram of total alkalinity versus total dissolved inorganic carbon, according to example implementation.

FIG. 10 is another vector slope diagram of alkalinity versus dissolved inorganic carbon with vector paths reflecting different materials used in the CDR reaction, according to example implementation.

FIG. 11 is an example of a computer architecture diagram of one implementation of the control system illustrated in FIGS. 1-3.

### DETAILED DESCRIPTION OF THE EMBODIMENTS

The following description of the embodiments of the invention is not intended to limit the invention to these embodiments but rather to enable a person skilled in the art to make and use this invention.

### Overview

Abiotic marine sequestration harnesses the chemical properties of the ocean to remove and store carbon dioxide (CO₂) from the atmosphere. In one approach, specific marine (including coastal, estuarine, and riverine outlet) waters that have excess biogenic CO₂ (from microbial respiration of dissolved organic matter) are targeted, removing atmosphere-bound CO₂ from the climate system. While much of the ocean system is under saturated in CO₂ relative to the atmosphere, thus absorbing CO₂ from the atmosphere, many regions in the global ocean system—such as some coastal waters or upwelling regions-are characterized by excess CO₂ (hereafter “high CO₂ waters”), and thus emit CO₂ to the atmosphere. Most of these high CO₂ waters are the result of microbial respiration of terrestrial—or marine-derived organic matter, with organic matter transport and water transport distance and speed generating hotspots for high CO₂ waters. The excess CO₂ of these waters causes localized acute acidification through the formation of carbonic acid. While the general systematics of organic matter respiration in water is natural, anthropogenic forcings, including land-use change and pollution, and reduced ocean circulation speed, are exacerbating these natural CO₂ emissions and acidification in some locations, to the detriment of climate stability and ecosystem health. Generally, high CO₂ waters are dynamic, with variable CO₂ emissions and acidification “events” and strong seasonal variability. In another approach, various waters (natural waters as above, or waste water streams or otherwise) can be leveraged for the capture and storage of external gaseous CO₂ (such as from point-source emissions, concentrated from point-source emissions or air, or from air directly). In both example approaches, the dissolved CO₂ in water can be used in this method to serve as a long-term storage sink for the CO₂ by incorporating the gaseous CO₂ into the water for subsequent conversion to bicarbonate and carbonate ions.

Systems and methods for a waterway environmental management system discussed herein function to enable an aqueous (or water) processing system. The systems and methods may have various environmental management capabilities within water/aqueous systems.

The systems and methods leverage an implementation whereby an input from a water source or some aqueous system (e.g., river, ocean water, industrial waste system) enters a management receptacle or container, a sensing system monitors a set of chemical conditions (e.g., measuring of CO2, HCO3, CO3) of the water, active introduction of additive is performed during monitoring (e.g., alkalinity is added while measurement tech measures the reaction (CO2->HCO3 or CO3)), active introduction of the additive is ended upon satisfaction of target chemical conditions, and a water or aqueous output is released to the water or aqueous system.

The systems and/or methods may involve a fully, partially, or un-submerged water treatment tank (i.e., a management receptacle), and sensor systems to determine CO2, HCO3, CO3 concentrations, pH, temperature, and other parameters (e.g., carbonate saturation state to determine the stability of the produced HCO3 and CO3). Outputs of the sensor systems may be used to determine the amount and rate of CO2 in the entering (pre-treated) waters and in the effluent or discharge of an output of treated waters, enabling a quantitation of the volume of CO2 captured and stored as non-gas carbon forms (e.g., bicarbonate and carbonate ions).

Herein, the systems and methods are primarily discussed as applied to water systems, where one or more management receptacles receives input water and discharges output water. However, the system may reasonably be modified for use on other aqueous systems as applicable such as waste systems from or within industrial processing systems.

Systems and methods are applicable to a continuous flow, or batch, or combined batch and continuous flow aqueous processing system that enables carbon dioxide reduction (CDR) from water (or from air or concentrated CO2 when percolated into this aqueous system). An example purpose of this system is to sequester large quantities of CO2 from various sources into bicarbonates and carbonates, including by adding alkaline elements (e.g., Mg, Ca, Na, K, etc.) to water as products of dissolution of alkalinity-containing minerals, brines, materials, or waters.

The systems and methods enable a water-based carbon storage solution for biogenic CO2 in natural waters, direct water capture, direct air capture, and/or point-source captured carbon using alkaline materials in water. In various embodiments, the systems and methods may be adapted to enable or facilitate solutions for direct air capture, carbon capture and storage, eutrophication/oxygenation management, and/or other applications of environmental system management.

This aqueous CO₂ removal and storage methodology actively manages and removes CO₂ in waters by manipulating the relative concentrations of the different species of dissolved inorganic carbon (CO₂, HCO₃, CO₃). In short, excess CO₂ is converted to HCO₃⁻ and CO₃²⁻ as a charge balancing reaction in response to the addition of alkalinity (e.g., Ca²⁺, Mg²⁺, Na⁺), expressed under the generalized and simplified equation 1 below. (Alkalinity is best defined as a conjugate base of a weak acid. Cations, or positively charged ions, are sometimes used as a definition for alkalinity, but not all sources (molecules containing cations) serve as functional alkalinity. For example, NaHCO₃ adds alkalinity because HCO₃⁻ is the conjugate base of a weak acid. But table salt (NaCl) does not add alkalinity because Cl⁻ is the conjugate base of a strong acid (HCl)).

This process reduces the aqueous pCO₂, either leading to less outgassing of CO₂ to the atmosphere from the water and thus less net CO₂ in the atmosphere over time, or leading to less emissions of the gaseous CO₂ incorporated into the water, driven by increased carbon storage in the ocean. This reaction neutralizes CO₂ by converting it to non-greenhouse gas dissolved inorganic carbon, effectively removing CO₂ from the atmosphere and storing it as HCO₃ and CO₃, which have residence times in ocean water reservoirs of 10,000-100,000 years.

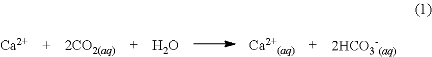 [SMILES: [Ca+2] ; O]

When performed correctly, the resulting, CO₂-neutralized waters are neither over-nor under-saturated in CO₂ with respect to the atmosphere; they are in equilibrium, at ˜425 ppm (˜0.5-0.7 mg/L), and thus should experience no subsequent change in carbon chemistry after the neutralization is performed, reducing uncertainty in potential down-stream effects (i.e., uncertainty in CO₂ uptake efficacy from air-sea gas exchange). Similarly, under some applications, when performed correctly, the resulting waters are undersaturated with respect to carbonate and similar minerals (e.g., aragonite), eliminating the potential for subsequent unintended chemical reactions in the water between the alkalinity and carbon which could precipitate the carbonate or similar minerals, driving a subsequent release of CO₂ (e.g., Ca2++CO32−″CaCO3). Under other applications, this method is used to intentionally drive the reaction (alkalinity and carbonate ion production) to be at or above carbonate or similar mineral saturation, to drive the precipitation of such minerals for various uses (e.g., CaCO3 mineralization).

The specifics of this approach were designed to generate “fully-measured carbon removal and storage,” wherein the reduction of CO₂, and subsequent increase in stable carbon storage products, dissolved or precipitated, are each individually quantified, eliminating uncertainties associated with other “natural environment” carbon removal and storage approaches. This can in some applications also result in a carbon removal approach that generates a net benefit to threatened ecosystems around the world by mitigating localized acidification.

In addition to dynamically modifying water systems, the systems and methods also enable accurately and consistently measuring aqueous carbon concentrations and other chemical conditions to more precisely and accurately control modifications. The measurement approach may control the containment duration, flow rate, and alkalinity dissolution speed, and addition of materials into water to: a) inhibit over-alkalinization (dissolution/addition of alkalinity) that would lead to precipitation of solid carbonate minerals, potential negative impacts on biological systems, and/or inefficient/overcostly use of additive, b) intentionally over-alkalinize, to intentionally lead to precipitation of solid carbonate minerals for additional uses, c) prohibiting precipitation of solid carbonate minerals (which could emit CO₂ in some aqueous systems), d) encourage precipitation of solid carbonate minerals (which could better store CO₂ in some systems), e) reduce alkalinity introduction when pre-treated water chemistry is low in CO₂ or high in alkalinity, and/or f) increase alkalinity introduction when pre-treated water is of higher CO₂/lower alkalinity. The measurement approach manages potential risk to the environment from water discharged back into the environment based on pH, temperature, the saturation state of carbonate materials, concentration of unreacted alkalinity, and other factors.

The systems and methods may be generally implemented as part of any closed or open water source for water monitoring and management. For closed systems, the systems and methods may be used to monitor and maintain water quality (e.g., reservoirs). For open systems, the systems and methods may enable fluid management in conjunction with other activities that occur on that body of water. For example, the systems and methods may be implemented in regions where waste is released, near mining operations, factory operations, heavy transport routes, and/or in other areas.

In one example, the systems and methods are used in connection with a water way such as a river, stream, or tidal estuary. The systems and methods may be used for monitoring and active adjustment of water conditions within the water way.

In another example, the systems and methods are used within an industrial water system, such as a mining operation. In this variation, the systems and methods are used for treating the water used in the mining operation and in some variations, also used for storing carbon that was captured from point-source emissions or ambient air (direct air capture).

In some variations, the systems and methods are used on land or above water (e.g., if this is placed on a ship or shore) in a container that holds water, allows flow-through of water, and/or discharges water into surface waters, soils, sediments, below-ground water reservoirs, groundwater, or other water treatment receptacles. The systems and methods may include different variations that can be used individually or in combination.

The systems and methods may provide a number of potential benefits and capabilities. The systems and methods are not limited to always providing such benefits and are presented only as exemplary representations for how the system and method may be put to use. The list of benefits is not intended to be exhaustive and other benefits may additionally or alternatively exist.

### Systems

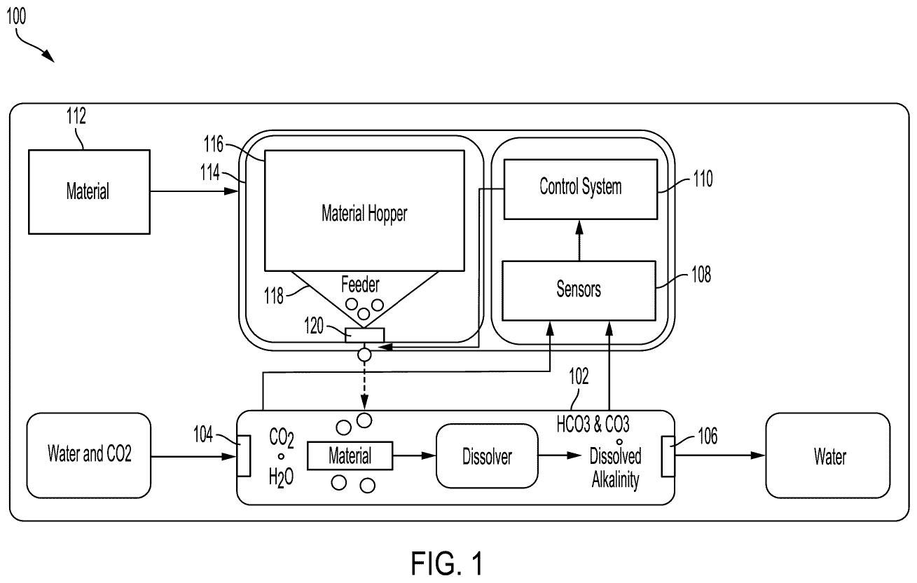

Referring now to the Figures, FIG. 1 illustrates a workflow diagram of an example of a system 100 for processing liquid to change an amount of carbon dioxide in the liquid, according to an example implementation. The system 100 includes a container 102 including an inlet 104 to receive liquid and an outlet 106 to release the liquid. The container 102 is for holding the liquid for processing. The system 100 also includes one or more sensors 108 coupled to the container 102 to measure carbon content in the liquid that is held in the container 102. The system also includes a control system 110 including a processor for executing instructions to: receive outputs of the one or more sensors 108, and based on the outputs of the one or more sensors 108, control dosing of material 112 into the container 102 that reacts with carbon dioxide to change the amount of carbon dioxide in the liquid such that an amount of the material 112 introduced into the container 102 changes over time responsive to changes in the carbon content being measured in the liquid.

The container 102 may take many forms, and in one example, holds liquid in large or small sizes depending on placement and configuration needs (as some examples, in a range of about 70 L of liquid for a small closed system to 2.5 million liters for a large closed system, e.g., size of swimming pool area). Thus, the container 102 functions as a tank or other form of receptacle where detection and/or treatment of water is processed. The container 102, when in an open state, allows the liquid to flow into and out of the container, and in a closed or operating state, prevents gaseous and liquid flow into or out of the management receptacle.

The container 102 receives liquid, such as water from a water source or some aqueous system (e.g., river, ocean water, industrial waste system). The inlet 104 and the outlet 106 are in communication (wired or wireless) with the control system 110, such that the control system 110 controls opening and closing of the inlet 104 and the outlet 106 based on signal transmission.

Thus, the inlet 104 is used to receive pre-treated water, and the outlet 106 is used to release water (which, following processing is treated water). In some variations, the inlet 104 and the outlet 106 are integrated to take advantage of a natural flow of a surrounding water system, such as a flow of a water river or stream or waves from a body of water. Thus, the inlet 104 and the outlet 106 can be the same defined opening through which water is received or discharged. In some variations, the inlet 104 and the outlet 106 have valves or doors that passively or actively control flow in and/or out.

The control system 110 is in communication with the inlet 104 and the outlet 106 to control opening and closing of the inlet 104 and the outlet 106.

The sensors 108 are shown coupled to the container 102 to receive samples of the liquid in the container 102. In an example implementation, illustrated in other figures described below, the sensors 108 are positioned inside the container 102 to measure various chemical conditions of the liquid (e.g., measuring of CO2, HCO3, CO3). The control system 110 is in communication (wired or wireless) with the sensors 102 to receive outputs of the sensors to determine amounts of carbon dioxide in the liquid.

The sensors 108 thus function to monitor a state of the fluid extracted from an external water system. In one example, the sensors 108 include a spectroscopic sensor and/or other sensors to measure properties of gas and/or liquid portions inside the container 102. The sensors 108 are situated to measure properties of an appropriate portion of the container 102 (i.e., gas sensors are situated in the gas portion and liquid sensors are situated in the liquid portion). In an example, the spectroscopic sensor is preferably attuned and positioned to measure carbon compounds in the gas portion of the measuring receptacle. More specifically, the spectroscopic sensor is attuned to detect and measure common carbon compounds in gas, such as carbon dioxide. In one example, the spectroscopic sensor is a non-dispersive infrared (NDIR) sensor.

In other examples, additional sensors are included such as a temperature sensor, a pressure sensor, a pH sensor, an alkalinity sensor, a flow meter, an electrical conductivity sensor, a refraction sensor (e.g., refractometer), a specific gravity sensor (e.g., hydrometer), and/or a hygrometer. Depending on the liquid for measurement and other environmental conditions, other sensors may be incorporated as well. The sensors 108 function to measure specific quantities of chemicals, and can work in conjunction to determine more complex fluid properties, such as viscosity and salinity.

The control system 110 is also in communication with an additive system 114 that is coupled to the container 102 including a repository of the material 112 (e.g., in a material hopper 116) to be added into the container 102. The additive system 114 includes a feeder 118 and a valve 120, and the control system 110 is in communication (wired or wireless) with the valve 120 to control operation of the valve 120 of the feeder 118 to release the material 112 from the feeder 118 into the container 102.

The additive system 114 functions to facilitate manipulation and/or modification of the liquid. Different variations of additives may be used. In one variation, the additive system 114 includes a repository of additive compound or material that may be deposited or otherwise added to a fluid. The additive system 114 may additionally include components to facilitate mixing. In some variations, multiple different additive compounds or materials are added (together or individually). In some variations, the system and/or the additive application system includes other components to facilitate modification of a water system.

In an example operation, the control system 110 controls active introduction of the material 112 into the container 102 to neutralize an amount of carbon dioxide in the liquid in the container 102. Thus, the control system 110 receives outputs of the one or more sensors 108, determines a carbon dioxide content of the liquid in the container 102, determines an amount of the material 112 to release into the container 102 in order to react with the carbon dioxide in the liquid, and then controls the valve 120 to do so. During introduction of the material 112 into the container 102, the sensors 108 continue to output data indicative of the chemical conditions of the liquid so that the control system 110 controls, in real time, amounts of additional material to add to the liquid.

In another example operation, the control system 112 controls opening and closing of the inlet 104 and the outlet 106 to control flow rate of liquid into and out of the container 102. The flow rate of the liquid into and out of the container 102 affects the carbon dioxide reaction with the material 112, such that a slower flow rate allows more carbon dioxide in the liquid to react with the material. As such, based on outputs from the sensors 108, the control system 112 modifies the flow rate accordingly (to optimize neutralization of CO₂ in the liquid) by controlling opening and closing of the inlet 104 and the outlet 106.

In still another example operation, the control system 112 controls opening and closing of the inlet 104 and the outlet 106 to control dwell time/duration of the liquid in the container 102. The duration of the liquid in the container 102 affects the carbon dioxide reaction with the material 112, such that a longer duration allows more carbon dioxide in the liquid to react with the material. As such, based on outputs from the sensors 108, the control system 112 modifies the duration of the liquid in the container 102 (to optimize neutralization of CO₂ in the liquid) by controlling opening and closing of the inlet 104 and the outlet 106.

The control system 110 thus functions to facilitate computer-controlled operation of the system 100. Various control approaches may be used to facilitate operation, as described herein. In some variations, the operation of one aqueous processing system may rely on data-driven operation based on external factors. In some variations, the operation of a plurality of aqueous processing systems may be coordinated such that they are operated as a network.

The system 100 may additionally include a power system. The power system may any suitable type of power system. In some variations, the power system may include or otherwise utilize current/tidal movement, river flow, gravity, solar photovoltaic (PV), and/or other non-renewable energy sources.

The material 112 includes powdered minerals, in one example, such as one of an alkaline element (e.g., magnesium (Mg), calcium (Ca), sodium (Na), potassium (K), etc.). Thus, the control system 110 monitors the chemical reaction (CO₂→HCO3+CO3) during active introduction of the additive.

In one example, the material 112 is alkaline feedstock sourced from a wide range of naturally occurring minerals and artificially synthesized materials, with the mineral and material matrix influencing the efficacy of the neutralization reaction based on its relative weight and charge. For example, calcium carbonate (CaCO₃), magnesium hydroxide (Mg(OH)₂), and magnesium silicate (Mg₂SiO₄) are all naturally occurring alkaline minerals that have different theoretical molar CO₂ neutralization capacities, reflected respectively in the stoichiometric equations 2-4.

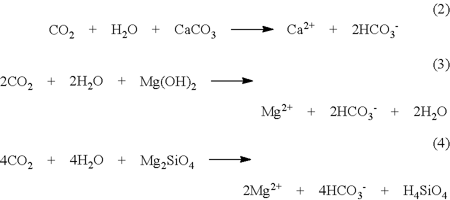

For a feedstock to be eligible for neutralization, it is beneficial to have the chemical composition to support the neutralization reaction (i.e., net alkaline), have the capacity for sufficient dissolution within the closed system, and meet conditions: input emission for synthesis, mining, transportation, and/or preparation must be less than the CDR capacity; and elemental composition analysis of potentially toxic elements (e.g., nickel, chromium) must demonstrate sufficiently low contaminant concentrations to support environmental safety.

In one example, the container 102 includes a dissolver (e.g., mixer) to assist with dissolving the material 112 in the liquid to increase the speed of conversion of carbon dioxide to HCO3 and/or CO3. In other examples, the material added includes pre-dissolved alkalinity based liquid, where the alkaline element is dissolved in water forming alkaline water that is then added to the liquid in the container 102.

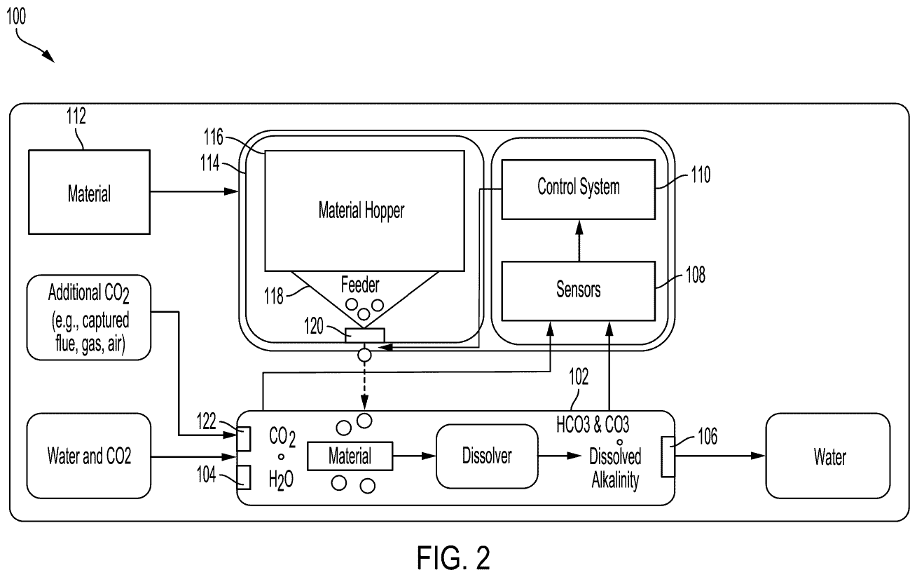

FIG. 2 illustrates a workflow diagram of another example of the system 100 for processing liquid to change an amount of carbon dioxide in the liquid, according to an example implementation. In FIG. 2, the container 102 includes the inlet 104 as a first inlet to receive liquid into the container 102, and a second inlet 122 to incorporate gas into the liquid within the container 102. Thus, the second inlet 122 is an air inlet to enable direct air capture (or other gases to enable carbon capture and storage (CCS) or carbon storage (CS)) into the container 102 to incorporate gas into the liquid within the container 102. In this example, the control system 110 receives outputs of the sensors 108, and based on the outputs of the sensors 108, controls dosing of the material 112 into the container 102 that reacts with carbon dioxide to change the amount of carbon dioxide in the liquid such that an amount of the material introduced into the container 102 changes over time responsive to changes in the carbon content being measured in the liquid. The control system 110 further controls the second inlet 122 to enable the direct air capture (or other gases) that incorporates air from any number of sources, such as external atmosphere, exhaust or flue gas from an emissions source, or storage that includes externally captured carbon dioxide. The air captured into the container 102 will be incorporated into the liquid in the container 102 to increase the carbon dioxide in the liquid. As such, the control system 110 then controls dosing of the material 112 into the liquid to neutralize the carbon dioxide in the liquid, for example, resulting in an overall effect of carbon capture.

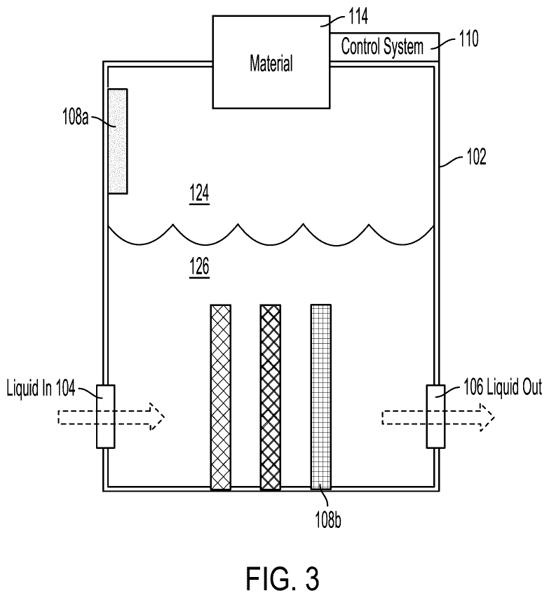

FIG. 3 illustrates an example of the system 100 in use, according to an example implementation. The system 100 functions as a monitoring device that once partially filled with the liquid, and in a closed state, detects and measures carbon content of the liquid. The closed state prevents liquid and airflow into or out of the container 102, and thus, the inlet 104 and the outlet 106 are closed in the closed state.

The container 102 is functionally divided into two portions: a top portion 126 (also referred to as a gas portion or non-filled region); and a bottom portion 124 (also referred to as the liquid portion or filled region). As used herein, the terms top portion and bottom portion (or gas and liquid portions) are generally functional designations that suggest a level that the container 102 is partially filled to function. That is, the top portion 124 and the bottom portion 126 do not necessarily suggest a different construction between the top portion 124 and the bottom portion 126, but an approximate level that the container 102 needs to be filled with a liquid to function. In some variations, these different regions may be separate.

In an example operation, the container 102 is partially filled with the liquid, and sensors 102a positioned in the top portion 124 measure gaseous carbon concentration measurements in the top portion 124 of the container 102. Other sensors 108b positioned in the bottom portion 126 measure general properties of the liquid (e.g., salinity, pH, and temperature). Outputs of the sensors 102a-b are used by the control system 110 to determine the liquid carbon concentration.

In another example, in FIG. 3, the top portion 126 is useful for air being percolated into the water in the bottom portion 124, such that the container 102 becomes entirely filled with water with a higher partial pressure of CO2.

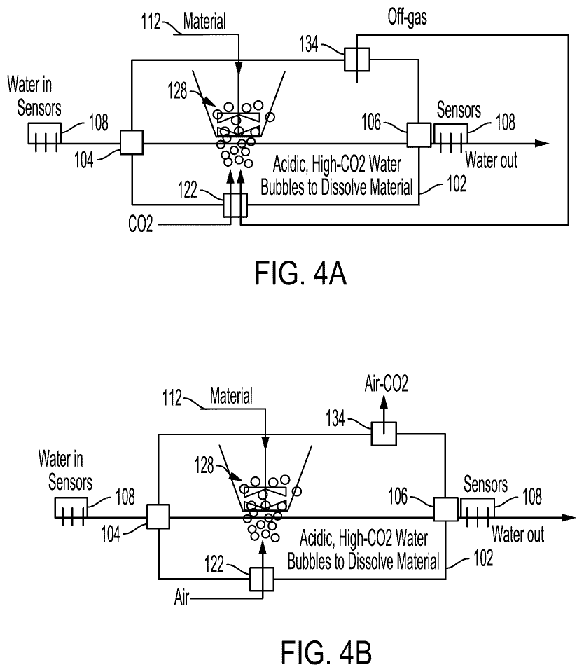

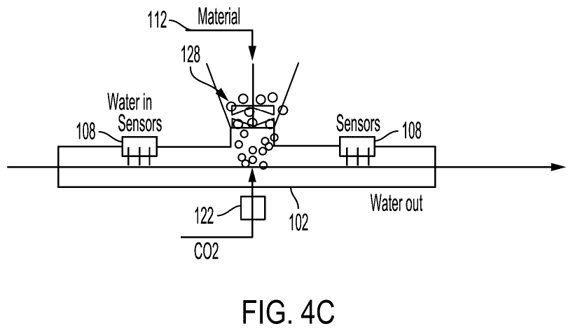

FIGS. 4A-4C are a set of schematic representations of different system variations for optimizing introduction of alkalinity and carbon for improved dissolution and carbon capture kinetics. In FIG. 4A, water is input and a first set of the sensors 108 measure carbon content of the water at the inlet 104. An amount of the material 112 is determined to optimize alkalinity and is combined through use of the mixer 128, in addition to introduction of high-CO₂ through the air inlet 122. In this example, alkalinity and CO2 are added together and increase in CO2 is measured and controlled. Off-gas is released through a vent outlet 134 and returned to the air inlet 122 for repeating the process. A second set of sensors 108 is adjacent to the outlet 106 to measure an amount of carbon content in the liquid being released from the container 102. Outputs of the first and second sets of sensors are compared to determine an amount of carbon converted to HCO3, for example.

In a further example, a sensor is positioned adjacent to the vent outlet 134 to measure the CO2 concentration in the off-gas to further assist with calculating a net change in CO2 in the liquid.

In FIG. 4B, an alternative is shown where air is introduced into the container, and the vent outlet 134 outputs off-gas into the external atmosphere.

In FIG. 4C, another alternative is shown, where input water is a continuous flow, and no air off gassing, but the container 102 is open to release treated water on a back end.

The system 100 is particularly useful for measuring and/or monitoring carbon compound concentrations in water. As a major contributor of carbon in water is carbon dioxide, the system 100 enables measuring and monitoring of carbon dioxide in water. In the same manner, the system 100 may be implemented to measure carbonate (CO3) and bicarbonate (HCO3) concentrations (or their conjugate acids, carbonic acid (H2CO3)). Generally, the system 100 may be implemented to measure fluctuations of these compounds (or other carbon compounds) in any fluid, and potentially determine the specific concentrations of each of these carbon compounds within the fluid.

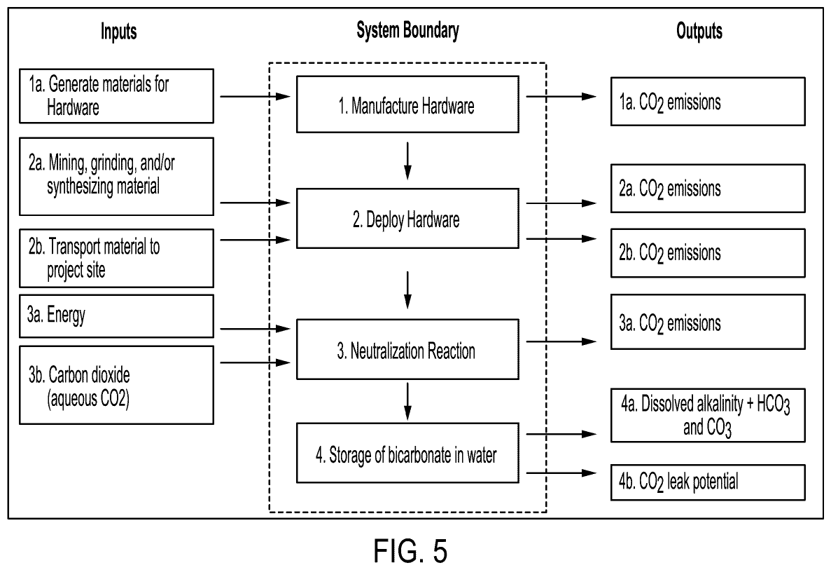

The monitoring capabilities or any similar monitoring capability may then be used in combination with various components for actively modifying a state of the water system. This may be used for different applications such as carbon storage, direct air capture, eutrophication, and/or other applications. Examples of different applications are described below with reference to FIGS. 5-11.

FIG. 5 is a block diagram illustrating inputs and outputs applicable to any of the systems illustrated in FIGS. 1-4, according to example implementations. Inputs include manufacturing of hardware (reactor, etc.) and associated materials (1a.), deployment of the hardware (including the acquisition, treatment, and transport of alkaline material) (2a.-2b.), and deployment of energy with aqueous biogenic carbon dioxide (3a.-3b.). Outputs include carbon dioxide emissions due to hardware manufacture (1a.), hardware deployment (2a.-2b.), and the neutralization reaction (including necessary energy inputs) (3a.), as well as, dissolved alkalinity, bicarbonate, and carbonate (4a.) and potential carbon dioxide leakage (4b.).

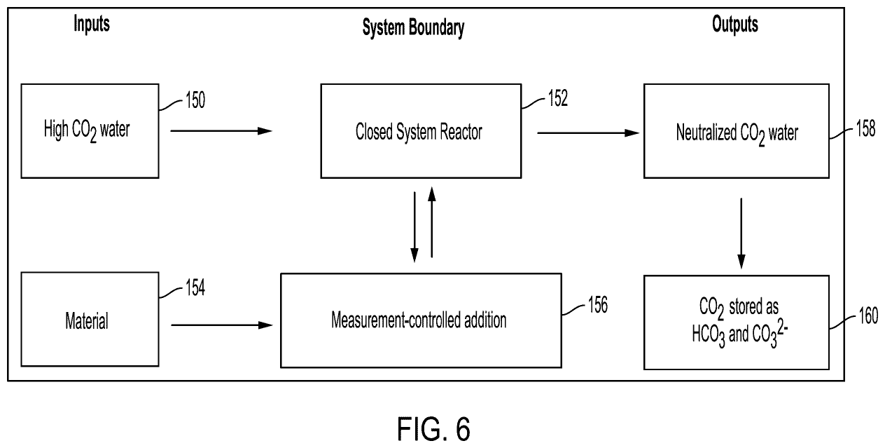

FIG. 6 is another block diagram illustrating the overall processing of water to generate CDR as performed by any of the systems illustrated in FIGS. 1-4, according to example implementations. Inputs of high carbon dioxide water 150 are provided to a closed system reactor 152, which may take the form of any of the systems illustrated in FIGS. 1-4. Next, alkalinity 154 (e.g., feedstock material 112 as shown in of the systems illustrated in FIGS. 1-4) is provided to a measurement-controlled addition mechanism 156 (e.g., the additive system 114, the mixer 128, or a combination of both, as shown in of the systems illustrated in FIGS. 1-4) for dosing of the high carbon dioxide water 150 in the closed system reactor 152. An output of the closed system reactor 152 is neutralized carbon dioxide water 158 and carbon dioxide captured/stored as bicarbonate and carbonate 160, for example.

### Processess—Measurement

The impact of added alkalinity is directly dependent on the input water carbon conditions—i.e., if there is no excess dissolved CO₂, no alkalinity should be added in this approach, as it will not lead to directly measurable neutralization of CO₂. Because natural carbon cycling in water is highly spatio-temporally dynamic and heterogeneous, this process benefits from direct, near- real-time measurement of baseline CO₂ mass (e.g., grams, moles) to control the release of alkalinity by mass based on both the input water and (when added) gas CO₂ concentrations.

Because multiple independent factors influence the CO₂ neutralization rate and efficacy, an example method to fully measure the CO₂ neutralization is to directly measure not only the starting CO₂ mass, but the CO₂, HCO₃ and CO₃ mass before and after the reaction. This enables (a) the measurement of the reduced dissolved CO₂ in response to the addition of alkalinity, independent of natural CO₂ reduction from degassing or photosynthesis, to assess additionality, (b) the generated HCO₃ and CO₃, independently demonstrating the reduction of CO₂ to stable (effectively “permanent removal”) carbon storage products as an additional assessment of additionality, (c) the efficacy of the added alkalinity (using molar ratios between alkalinity and carbon) to determine to what degree the reaction truly is “fully measured” vs how much remaining alkalinity may be in the water un-reacted with CO₂, and (d) the resulting ecological impacts in carbon space, such as carbonate saturation state, to quantitatively assess the potential leakage and ecosystem impacts.

Additional factors such as temperature, salinity, baseline total alkalinity, and total dissolved inorganic carbon (the combined concentrations of CO₂, HCO₃, and CO₃) directly control the efficacy of alkalinity addition on CO₂ neutralization. On top of these factors controlling the intervention of alkalinity addition, natural CO₂ concentrations are modified in water via biological (i.e., photosynthesis, respiration, calcification) and physical (e.g., temperature-induced degassing, wind-driven mixing) drivers.

Within some examples, a closed system is required to perform these measurements effectively, such that the concentrations of carbon compounds (mg/L) can be used to determine the mass (mg) based on the closed system volume (L). Due to the closed system, the concentrations of carbon compounds and volume of water determine the mass of alkalinity. Upon full interaction between alkalinity and carbon, because of the lack of dilution or transportation, final carbon compound concentrations can be quantified. Within other examples, a continuous flow system can perform these measurements effectively by combining measurements of the concentrations of carbon compounds (mg/L) can be used to determine the mass (mg) through time based on the flow rate of the water through the system (L/min).

The closed system, measurement platform, and alkaline addition mechanisms combine to require a physical hardware system (“Hardware”) that is be manufactured, installed, and operated in proximity to high CO₂ water.

Emissions associated with this approach include (1) CO₂ emissions from the water naturally (baseline greenhouse gas (GHG) emissions), (2) GHGs emitted from the generation of the alkaline feedstock which is used to drive the CO₂ neutralization, including (depending on the feedstock being generated) mining and grinding (for mineral-derived alkalinity), including mineral separation and energy inputs (for electrochemically-derived alkalinity), (3) GHGs emitted from the transportation of the alkaline feedstock, (4) GHGs emitted from the production of energy used in- situ during the entire CO₂ neutralization process, including: additional processing of the alkaline feedstock before addition to water (e.g., energy inputs for dilution or dissolution, energy inputs for mechanical addition of alkalinity to water), energy inputs for moving water in and out of the closed system, energy inputs for the measurement, energy inputs for Hardware manufacturing (i.e., materials like pumps, alkaline holding and closed system tank, etc., as well as direct manufacturing emissions), amortized over gross CO₂ reduction, and (5) potential GHG emissions from the water upon release from the closed system (e.g., conversion of stable HCO₃ or CO₃ back to CO₂ via runaway precipitation; AKA leakage).

Near-real-time measurements of the starting and ending carbon concentrations, coupled with a volumetric measurement from the closed system, enables calculation of net carbon removal (Equations 5-7):

$$Gross Reduced {CO}_{2} ( g ) = \sum ( Baseline {CO}_{2} ( mg / L ) - Post Treatment {CO}_{2} ( mg / L ) ) * L \qquad ( 5 )$$

$$Bicarbonate Generated ( g ) = \sum ( Post Treatment {HCO}_{3} ( mg / L ) - Baseline {HCO}_{3} ( mg / L ) ) * L \qquad ( 6 )$$

$$Carbonate Generated ( g ) = \sum ( Post Treatment {CO}_{3} ( mg / L ) - Baseline {CO}_{3} ( mg / L ) ) * L \qquad ( 7 )$$

Until the storage products HCO₃ and CO₃ are quantitatively accounted for, measured CO₂ decrease is referred to as a reduction—i.e., there is less in the outgoing waters than there was in the starting waters. This reflects an inability to trace the actual fate of that CO₂ based just on the CO₂ measurement so that when the resulting storage products (HCO₃ and CO₃) are accounted for, this can be referred to as “removal and storage.”

Operated under continuous flow conditions, flow rate measurements within the closed system are used to calculate volume within project timeframes as in Equation (8).

Treated volume of water (L)=Flow rate (L/min)*Time of project operation (min) (8)

To assess how much of the CO₂ reduction measured was due to alkalinity-driven neutralization to stable inorganic carbon (rather than degassing or biological processes), a final efficacy calculation (Equation (9)) is performed using the molar equivalents of each carbon compound:

$$Efficacy ( \% ) = ( ( Bicarbonate Generated ( mol ) + Carbonate Generated ( mol ) / Gross Reduced {CO}_{2} ( mol ) ) * 100 \qquad ( 9 )$$

Thus, the total gross additional CO₂ reduction is reported as either Equation (10) or (11), with the two being equivalent:

$$Total Gross Additional Removed {CO}_{2} ( g ) = Gross Reduced {CO}_{2} ( g ) * ( Efficacy ( \% ) / 100 ) \qquad ( 10 )$$

$$Total Gross Additional Removed {CO}_{2} ( g ) = ( Bicarbonate Generated ( mol ) * ( 44 / 61 ) ) + Carbonate Generated ( mol ) ( 44 / 60 ) ) * 44 \qquad ( 11 )$$

From there, the Net Removed CO₂ is calculated accounting for the additional system boundary conditions using Equation (12) based on a completed Life Cycle Assessment (LCA):

$$Total Net Additional Removed {CO}_{2} ( g ) = Total Gross Additional Removed {CO}_{2} ( g ) - Total {CO}_{2} e ( g ) from alkaline feedstock generation - Total {CO}_{2} e ( g ) from alkaline feedstock transportation - Total {CO}_{2} e ( g ) from in - situ process energy use ( \sum ( Total {CO}_{2} e ( g ) from additional alkaline feedstock processing ) + ( Total {CO}_{2} e ( g ) from water movement energy use ) + ( Total {CO}_{2} e ( g ) from direct measurement energy use ) + ( Total {CO}_{2} e ( g ) from Hardware manufacturing / total {CO}_{2} reduction ( g ) generated by the Hardware ) ) - Total {CO}_{2} e ( g ) from ex - situ {CO}_{2} loss ( leakage ) \qquad ( 12 )$$

These LCA factors are assessed in the following ways:

Total CO₂e (g) from alkaline feedstock generation=The mass of feedstock added throughout the duration of the CO₂ neutralization (g)*The emissions produced by the generation of that particular alkaline feedstock (g CO₂e/g feedstock). The latter can be found from existing repositories (i.e., for CaCO₃, US Dept. of Energy Emissions Factors for limestone mining), but ideally is directly measured from the alkaline generation source.

Total CO₂e (g) from alkaline feedstock transportation=The mass of feedstock added throughout the duration of the CO₂ neutralization (g)*The emissions produced by the transport of that feedstock from the specific generation site to the deployment site (g CO₂e/g feedstock). This can be derived from reported emissions factors generalized by transportation method (e.g., diesel engine emissions, from the US Energy Information administration Emissions Factors for diesel combustion engines, accounting for total mass of material transported per dump truck); while ideally these emissions are measured directly (i.e., at exhaust point of diesel engine), such direct measurement at scale is unlikely. However, direct measurement of mass of feedstock transported (e.g., grams of minerals loaded per truck) should be known given transportation weight standards.

Total CO₂e (g) from additional alkaline feedstock processing, Total CO₂e (g) from water movement energy use, and Total CO₂e (g) from direct measurement energy use may be measured and calculated individually, but practically, these are combined as a single in-situ project energy use CO₂e (g) monitored over the course of the CO₂ neutralization. Because these are from a single project, the energy use is calculated based on the source of energy (e.g., gas generator, solar panel). These are then combined with the amortized Total CO₂e (g) from manufacturing the Hardware, which cannot be directly measured—the combination of specific manufactured parts that go into the Hardware leads to a practical supply chain reporting issue wherein total input emissions must be estimated based on key factors (e.g., total amount of steel used (g)*steel manufacture emissions (CO₂e g/g steel)). These Hardware emissions, to be amortized, are then divided by the total gross CO₂ neutralization mass (g) generated by the Hardware. A conservative approach is to use the actualized gross CO₂ neutralization mass (g) (i.e., how much CO₂ the system has already removed) to amortize Hardware emissions. This approach makes the net Hardware input emissions much higher initially, decreasing over time. However, this approach is significantly biased against early tonnage—the initial tonnage produced by a Hardware would simply be used to counteract the input emissions before net CO₂ removal and storage is generated for offset sales. In other words, a Hardware would need to offset its own manufacturing emissions before generating any net CO₂ removal volume to be sold or reported as net offsets or insets. An alternative potential approach that may be acceptable to buyers is to use an estimate of the total CO₂ removal a Hardware unit will generate over its lifetime—i.e., an assumption that a system will be online for X amount of time, converting on average Y mass of CO₂ per unit time, thus yielding a total Z mass of CO₂ over the system lifetime. In this approach, the total Hardware emissions would be divided by that Z mass until that Z mass of CO₂ has been converted by the system, after which the Total CO₂e (g) from manufacturing the Hardware would be 0 g.

Total CO₂e (g) from ex-situ CO₂ loss (leakage) is estimated rather than directly measured, because upon exiting the closed system and experiencing transportation and dilution, the dissolved carbon compounds will elude direct measurement. Three methods can be leveraged (ideally together) to quantitatively assess these emissions: (a) a vector slope assessment described below can demonstrate an excess mineral dissolution relative to precipitation using the in-situ measurements within the system, (b) the final conditions of the water before release from the closed system can be used to estimate downstream fate (e.g., the carbonate saturation state and pH can elucidate whether or not the water may experience later carbonate saturation, using a two-box model wherein the starting water concentration is assumed to be the condition of the ambient water into which the closed system drains), and (c) more refined basin-scale numerical geospatial modeling can be used with independent downstream sensors and measurements to assess how the resulting ex-situ water chemistry has been changed. Because this overall approach leaves water at equilibrium with atmospheric CO₂, the resulting pH and carbonate saturation state should not result in any precipitation or leakage, and thus, this value should by virtue of the approach be negligible. In cases where mineral precipitation is the target, the measurement of output aqueous carbon concentrations can determine the amount of CO₂ stored as mineral and amount of CO₂ remaining in aqueous form for potential leakage from the water back to the atmosphere.

### Processess—Measurement Verification

Example measurement approaches described herein enable use of real time dissolved carbon inputs and outputs to directly quantify and control the permanent storage of CO2 gas as dissolved HCO3 and CO3. In-situ dissolved carbon data generated by the control system 110 and the sensors 108 is gathered at the input and output of the system, with associated input emission data used to calculate net CO₂ stored. A novel approach to calculating mineral dissolution and carbonate saturation states from the data enables the demonstration of dissolved HCO3 and CO3 permanence and CO₂ storage process efficiency. These outputs are compared quantitatively to target water chemistry outputs and used accordingly to control key process input rates.

Examples thus include planning and monitoring, reporting, and verification (MRV) requirements for use in offsetting and insetting project development to generate high-quality carbon dioxide removal and storage (CDR) or carbon dioxide capture and storage (CCS) credits through carbon dioxide (CO₂) neutralization in natural waters.

An example CDR approach, described herein, uses a closed system to neutralize CO₂, i.e., remove CO₂ in high-CO₂ water until it is in equilibrium with the atmosphere. This neutralization is done by converting aqueous CO₂ to bicarbonate and carbonate through a charge-balanced reaction facilitated by the measurement-controlled addition and active dissolution of alkaline minerals. This generates a CDR or CCS credit that is: functionally permanent (given bicarbonate and carbonate have residence times in water reservoirs of over 10,000 years), immediate (as the CDR reaction occurs within the project system boundary), and measurable (as a closed system project boundary with rapid reaction times enables the direct quantification of incoming CO₂ and outgoing CO₂ and resulting produced bicarbonate and carbonate).

The physical setup of the system can take many forms, but generally includes: (1) a closed-loop reactor pipe through which water flows uni-directionally, isolated from external water sources except at the designated inlet, and in this reactor alkaline minerals are introduced via a dosing mechanism downstream of an initial CO₂ measurement so that the reactor facilitates the dissolution of these minerals; and (2) two identical measurement setups to analyze the carbonate system and other parameters at the inlet (baseline) and outlet (post-treatment).

Measuring not only the change in CO₂ but the full carbonate system before and after the reaction, enables several benefits for the measurement verification approach. First, this confirms that the reduced dissolved CO₂ is in response to the addition of alkalinity, independent of short-term CO₂ changes. Next, this demonstrates that the bicarbonate and carbonate generated were the result of the neutralization of CO₂ to stable carbon storage products (effectively “permanent removal”). In addition, this assesses the efficacy of the added alkalinity (using molar ratios between alkalinity and carbon) to determine to what degree the reaction truly is “fully measured” versus how much remaining alkaline minerals may be in the water unreacted with CO₂. Still further, this assesses impacts on carbon systematics, such as carbonate saturation state, to assess the potential leakage and ecosystem impacts quantitatively.

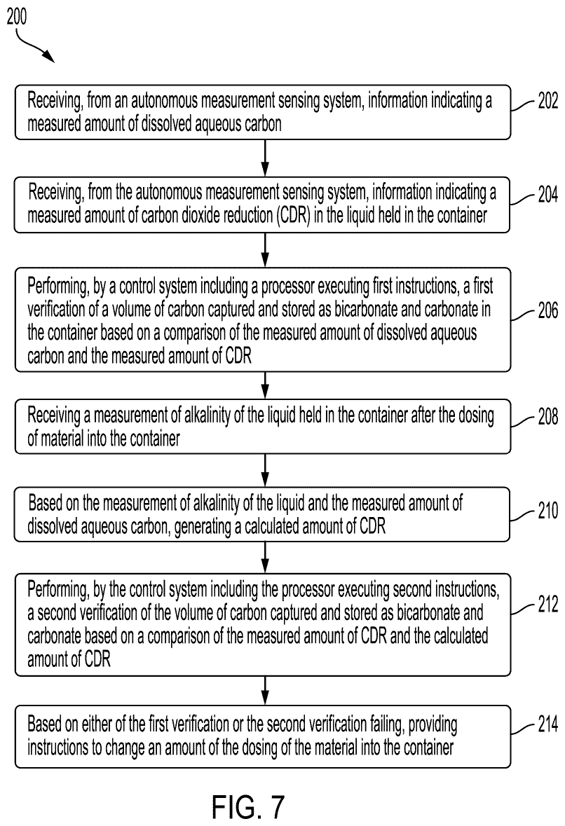

FIG. 7 shows a flowchart of an example of a method 200 for quantifying an amount of carbon dioxide captured from liquid, according to an example implementation. Method 200 shown in FIG. 7 presents an example of a method that could be used with or implemented by the system 100 shown in FIGS. 1-4, the control system 110 in FIGS. 1-3, or a separate and independent control system/computing device in communication with the system 100 or the control system 110 via wired or wireless communication (or over network communications), for example. Thus, the system 100, when performing the functions of the method 200, is a system for quantifying an amount of carbon dioxide captured from liquid, for example.

Further, devices or systems described herein may be used or configured to perform logical functions presented in FIG. 7. In some instances, components of the devices and/or systems may be configured to perform the functions such that the components are actually configured and structured (with hardware and/or software) to enable such performance. In other examples, components of the devices and/or systems may be arranged to be adapted to, capable of, or suited for performing the functions, such as when operated in a specific manner. Method 200 may include one or more operations, functions, or actions as illustrated by one or more of blocks 202-214. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

It should be understood that for this and other processes and methods disclosed herein, flowcharts show functionality and operation of one possible implementation of present examples. In this regard, each block or portions of each block may represent a module, a segment, or a portion of program code, which includes one or more instructions executable by a processor for implementing specific logical functions or steps in the process. The program code may be stored on any type of computer readable medium or data storage, for example, such as a storage device including a disk or hard drive. Further, the program code can be encoded on a computer-readable storage media in a machine-readable format, or on other non-transitory media or articles of manufacture. The computer readable medium may include non-transitory computer readable medium or memory, for example, such as computer-readable media that stores data for short periods of time like register memory, processor cache and Random Access Memory (RAM). The computer readable medium may also include non-transitory media, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a tangible computer readable storage medium, for example.

In addition, each block or portions of each block in FIG. 7, and within other processes and methods disclosed herein, may represent computing device(s) and/or circuitry that is wired to or adapted to perform the specific logical functions in the process. Alternative implementations are included within the scope of the examples of the present disclosure in which functions may be executed out of order from that shown or discussed, including substantially concurrent or in reverse order, depending on the functionality involved, as would be understood by those reasonably skilled in the art.

At block 202, the method 200 includes functions of receiving, from an autonomous measurement sensing system, information indicating a measured amount of dissolved aqueous carbon. The measured amount of dissolved aqueous carbon is generated by dosing of material into a container holding the liquid that reacts with carbon dioxide in the liquid. In one example, the autonomous measurement sensing system includes the sensors 108 of the system 100. In another example, the autonomous measurement sensing system includes the sensors 108 and the control system 110 of the system 100. The autonomous measurement sensing system thus includes sensors capable of performing described measurements, for example. The measured amount of dissolved aqueous carbon is determined using outputs of the sensors 108, and using any or all of Equations (5)-(11) above.

Note that, in some instances, carbonate and bicarbonate may not be directly measured, but indirectly inferred from other carbonate system parameters using sensing methods. Thus, in some examples, information indicating the measured amount of dissolved aqueous carbon includes estimates of the amount of dissolved aqueous carbon.

At block 204, the method 200 includes functions of receiving, from the autonomous measurement sensing system, information indicating a measured amount of carbon dioxide reduction (CDR) in the liquid held in the container. In one example, the measured amount of CDR is calculated by measurements of the liquid (based on outputs of the sensors 108) at an input or inlet 104 to the container 102 pre-treatment and the liquid at an output or outlet 106 of the container 102 post-treatment after dosing of the material into the container for reaction with the carbon dioxide in the liquid (see, e.g., FIGS. 4A-4C). The measured amount of CDR is the difference in the pre-treatment and post-treatment measurements.

At block 206, the method 200 includes functions of performing, by a control system including a processor executing first instructions, a first verification of a volume of carbon captured and stored as bicarbonate and carbonate in the container based on a comparison of the measured amount of dissolved aqueous carbon and the measured amount of CDR. In one example, performing the first verification includes determining whether the measured amount of dissolved aqueous carbon and the measured amount of carbon dioxide reduction (CDR) are approximately equal (e.g., approximately equal in terms of molar equivalents). Approximately includes deviations from exactness covering rounding errors and deviations of amounts in a range of 0-1%, 0-2%, or 0-3%, for example. Approximately also includes, in other examples, a larger percentage range (e.g., 5-10%) when amounts in comparison are of larger magnitudes.

In one example, as described in Equation (9) above, molar mass equivalents are used for such verification, with an example of a measured 1 tonne of dissolved aqueous carbon being verified as CDR through measured 1.39 tonnes of dissolved bicarbonate. Note that the molar mass of CO2 is 44g/mol and HCO3 is 61 g/mol, so by this method of demonstrating the CO2 removed and CO2 stored is the same, a comparison of units should be in terms of molar equivalents, which is a different mass. In this instance, 1 tonne of CO2 removed will show up as 1.39 tonnes bicarbonate generated. Thus, within some examples, verification herein considers approximately equal in terms of moles/atoms and the calculation allows that assessment.

At block 208, the method 200 includes functions of receiving a measurement of alkalinity of the liquid held in the container after the dosing of material into the container. The measurement is output from one of the sensors 108.

At block 210, the method 200 includes functions of based on the measurement of alkalinity of the liquid and the measured amount of dissolved aqueous carbon, generating a calculated amount of CDR. In one example, Equations (1)-(4) above are used to calculate the amount of CDR.

At block 212, the method 200 includes functions of performing, by the control system including the processor executing second instructions, a second verification of the volume of carbon captured and stored as bicarbonate and carbonate based on a comparison of the measured amount of CDR and the calculated amount of CDR. In one example, performing the second verification includes determining whether the measured amount of carbon dioxide reduction (CDR) and the calculated amount of carbon dioxide reduction (CDR) are approximately equal. Approximately, again, has meaning as previously described.

At block 214, the method 200 includes functions of based on either of the first verification or the second verification failing, providing instructions to change an amount of the dosing of the material into the container. Instructions are provided to the control system 110 (or determined by the control system 110) to change an amount of the dosing of the material into the container.

Within examples, failing of the first verification or the second verification includes the compared amounts not be approximately equal, or being outside of an acceptable amount of deviation from one another. The verifications are performed to establish the truth or validity of the measurements. When a verification fails, one of the measurements is more than likely not accurate or it may be possible that an error has occurred in the system. Additionally or alternatively, an amount of deviation (causing the verification to fail) can be used to report an uncertainty of the carbon credit as well.

In some examples, functions of the method 200 also include based on either of the first verification or the second verification failing, triggering shut-down of the dosing of the material into the container via communication with the control system 110 configured to control the dosing.

In some examples, functions of the method 200 also include based on either of the first verification or the second verification failing, requesting from the autonomous measurement sensing system a second set of measurements indicating an updated measured amount of dissolved aqueous carbon and an updated measured amount of carbon dioxide reduction (CDR) in the liquid held in the container. In this manner, the measurements are re-tested for verification.

In some examples, functions of the method 200 include quantifying an amount of volatile impurities in the liquid that impact the volume of carbon captured and stored as bicarbonate and carbonate in the container. The verification functionality extends to point-source CO2 capture in mixed gases. If a mixed gas contains volatiles other than CO2, the verification functionality correctly determines neutralization of all species, including CO2. The end product is used to support a calculation in which neutralizing additional volatiles is estimated separate from neutralizing CO2. In one example, this can both increase the certainty of the credited CCS/CDR, and be used to appropriately then increase or decrease the alkalinity added to ensure complete neutralization of the CO₂ on top of neutralization of those other volatile species. For example, SOx+CO2 in a gas stream introduced into water are both neutralized by alkalinity—this method allows the alkalinity to be adjusted to neutralize both, and a calculation of the relative neutralization of both such that only the CO₂ neutralized is credited.

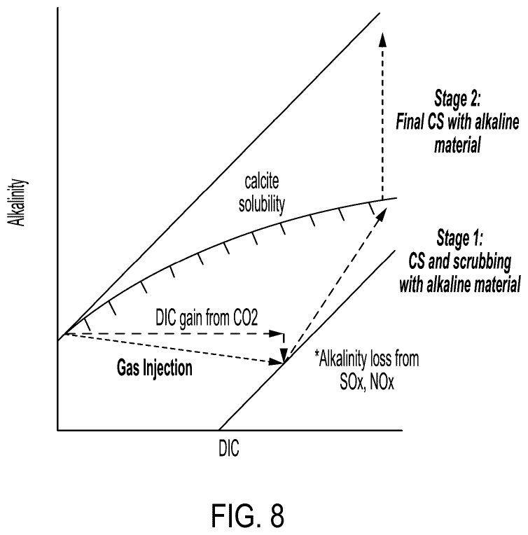

FIG. 8 is a graph illustrating alkalinity versus dissolved inorganic carbon (DIC), according to an example implementation. In FIG. 8, alkalinity increases linearly with increases in DIC. Gas injection of clean gas (no NO2, SO2, etc.) should add DIC but not alkalinity. When gas is injected (see, e.g., FIGS. 4A-4C), if CO2 is the only volatile, then alkalinity will be about constant. However, if additional volatiles (e.g., SO2 or NO2) enter the water, the resulting strong acids decrease alkalinity. In FIG. 8, even if flue gas is scrubbed below regulatory limits (two-stage process), some volatiles may remain.

Thus, in FIG. 8, the Gas Injection line will be horizontal across for CO2 as the only volatile, or downward sloping (as shown in FIG. 8) when extra volatiles are present. The loss of alkalinity (along the y-axis) is translated to a presence of impurities (other than carbon dioxide) in the water.

The verification functionality works using the sensor system to calculate alkalinity after gas injection (see, e.g., FIGS. 4A-4C) but before mineral addition. If CO2 is the only volatile, then alkalinity will be constant. However, if additional volatiles (e.g., SO2 or NO2) enter the water, the resulting strong acids decrease alkalinity. The alkalinity “deficit” is balanced by alkaline feedstock addition, which is accounted separately from feedstock used to neutralize CO2. The accreditation process relies on this distinction. Any feedstock that neutralizes H2SO4 and HNO3 does not reduce CO2, and cannot be counted toward carbon reduction.

Thus, in some examples, the functions of method 200 include receiving a first initial measurement of alkalinity of the liquid held in the container prior to dosing of material into the container, receiving a second initial measurement of alkalinity of the liquid held in the container after injection of carbon dioxide into the container and still prior to dosing of material into the container, and based on the first initial measurement of alkalinity and the second initial measurement of alkalinity being approximately equal, quantifying an amount of volatile impurities in the liquid as nominal. When the first initial measurement of alkalinity and the second initial measurement of alkalinity are not approximately equal, the amount of volatile impurities in the liquid is more than nominal.

In further examples, functions of the method 200 include based on the second initial measurement of alkalinity being less than the first initial measurement of alkalinity by a threshold amount, determining an amount of additional dosing of the material into the container that is required to balance the alkalinity to a desired level, determining an amount of bicarbonate and carbonate generated by the additional dosing of the material into the container, and separately accounting for the amount of bicarbonate and carbonate generated by the additional dosing of the material into the container to balance alkalinity of the liquid and the volume of carbon captured and stored as bicarbonate and carbonate in the container based on the dosing of the material to neutralize carbon in the liquid.

Functions of the method 200 adopt a conservative estimation of the CDR outcomes, in some examples, through a number of possible measures. CDR calculations, in one example, only account for reduced outgassing of CO₂ and not potential for additional absorption of CO₂ from the atmosphere by the treated water as a result of the reduced net CO₂ concentration of the water body. Thus, only measured CO₂ neutralization is accounted for as CDR. Any potential for subsequent uptake of CO₂ from the treated waters is not accounted for.

In another example, CDR calculations rely only on measured CO₂ neutralization. This is a more conservative approach than model-driven MRV and calculations, which may assume a number of outcomes (e.g., full dissolution of solid alkaline minerals) that could overestimate carbon impacts.

In still another example, CDR calculations incorporate conservative estimates of uncertainties based on independent validations. That cumulative analytical uncertainty is subtracted from the gross CO₂ calculation (i.e., only the lower bound of the measured CDR is considered).

In still another example, only CO₂ reduction that is correlated with a concomitant increase in bicarbonate and carbonate is considered as a CDR under this methodology, given the multiple non-CDR drivers that could lead to a reduction in dissolved CO₂. When the resulting storage products (bicarbonate and carbonate) are accounted for, then this in good faith is referred to as “removal and storage”. This is quantitatively accounted for in Equation (9) above.

In yet a further example, baseline CO2 is set at a minimum of 500 ppm, rather than exactly at equilibrium with mean global atmospheric concentrations (i.e., closer to 425 ppm), to account for local atmospheric CO₂ that could be above 425 ppm, ensuring that the calculated net CDR is representative of CO₂ removed from the climate system and stored.

In some examples, evidence of mineral dissolution is generated to demonstrate that the CO₂ conversion is indeed an “additionality” effect of the CDR process, and not driven by unintended, short-term changes in carbon cycling within the closed system reactor. Here, a method to generate quantitative evidence that the measured changes are driven by mineral dissolution is also described.

Mineral dissolution can be assessed using vector paths on a Deffeyes diagram, where relative changes in total dissolved inorganic carbon and total alkalinity (TA) can be interpreted as reaction pathways reflective of the driver of observed changes in aqueous carbon cycling. Thus, the relative change in TA and total dissolved inorganic carbon can be used to demonstrate not only whether the measured carbon cycle changes associated with this CDR approach were driven by mineral dissolution, but which mineral was dissolved to drive the observed change.

To use this method, pH and the sum of CO₂, bicarbonate, and carbonate (collectively, total dissolved inorganic carbon) are used to calculate TA. When compared between inlet and outlet waters, vector slope is indicative of the cause of carbon cycle changes.

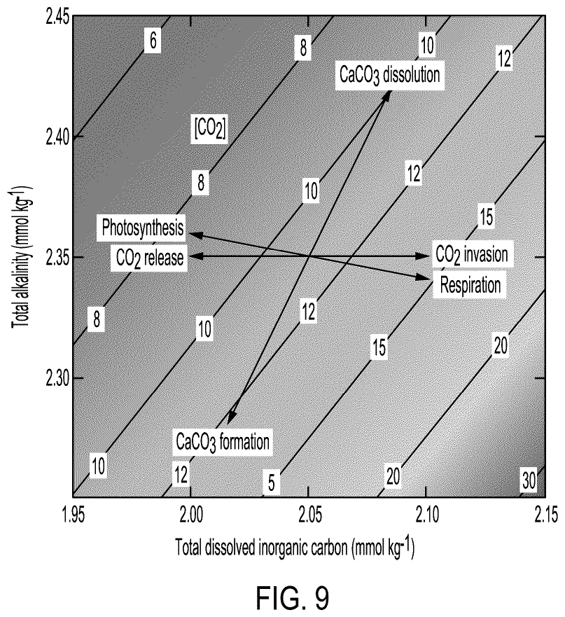

FIG. 9 is a vector slope diagram of total alkalinity versus total dissolved inorganic carbon, according to example implementation. In FIG. 9, for example, directional vectors (with quantitative slopes) indicate drivers such as photosynthesis, CaCO₃ precipitation, or mineral dissolution, as labeled. The Deffeyes diagram of total dissolved inorganic carbon and TA shows the vector paths reflecting reaction pathways and associated drivers of carbon cycle changes.

Using the inlet and outlet water measurements described here, this vector slope approach can quantitatively demonstrate that (a) changes in carbon cycling showing CDR are attributable to effective mineral addition, and (b) which mineral type specifically was used to drive the CDR.

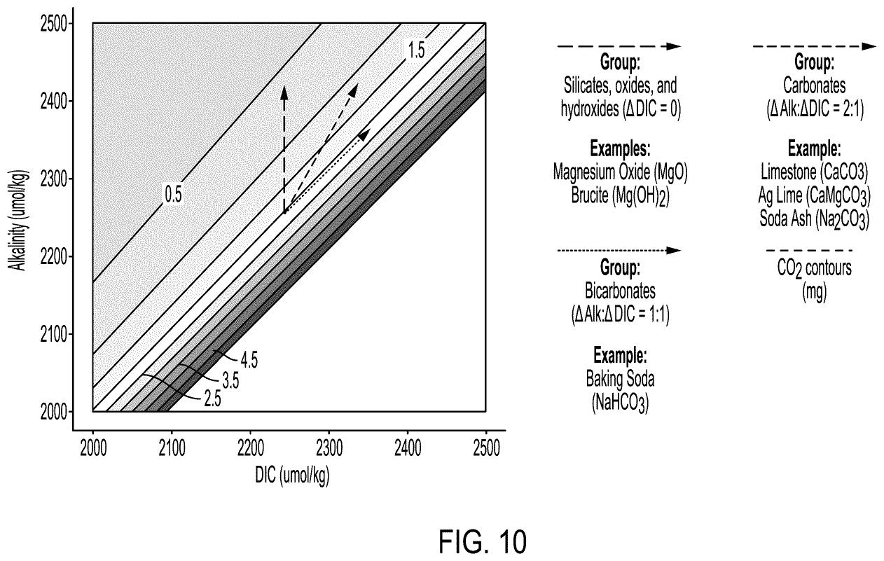

FIG. 10 is another vector slope diagram of alkalinity versus dissolved inorganic carbon with vector paths reflecting different mineral compositions, according to example implementation. In FIG. 10, the vector slope quantification approach differentiates the in-situ dissolution of different minerals. For example, an undefined slope (vertical line) is indicative of mineral matrices without carbonate or bicarbonate components (e.g., hydroxides, oxides, and silicates), a slope of about 2:1 is indicative of carbonate (CO₃²⁻-bound) minerals, and a slope of about 1 is indicative of bicarbonate (HCO₃⁻-bound) minerals. Also, vectors of oxygenic photosynthesis and respiration (i.e., carbon exchange in short-term carbon pools that are not considered durable CDR pathways) have very different vector paths.

In one example, this can be applied as a time series where inlet and outlet water measurement comparisons are vectorized and averaged (or through-time) slopes reported as:

$$Vector Slope = [ TA {( μmol / kg )}_{outlet} - TA {( μmol / kg )}_{inlet} ] / [ DIC {( μmol / kg )}_{outlet} - DIC {( μmol / kg )}_{inlet}$$

### Processess—Measurement Validation

In some examples, components of measurement verifications are further independently validated by third-parties to calibrate net CDR measurements and to confirm key assumptions around externalized impacts. Because safety and efficacy of this process rely on, and are assessed using, in-situ near real time measurements, the in-situ near-real-time measurements are validated using independent traditional analytical procedures (e.g., coulometric or potentiometric titrations) by an independent third-party. Water is sampled from the inlet and outlet of the closed system, timestamped to a corresponding in-situ measurement, and analyzed for the same components measured in-situ (i.e., pH, CO₂, HCO₃, CO₃). The resulting analytical uncertainty (%) between the in-situ and laboratory analyses is propagated between sampling intervals (i.e., each month), and the lower-bound CO₂ neutralization calculations (CO₂ removed, HCO₃ and CO₃ generated) ascribed to define the corrected gross measurements as in equations (13-15). Those are then used throughout the equations as conservative, independently-validated measurement values.

$$Sensor - Corrected Gross Reduced {CO}_{2} ( g ) = ( Gross Reduced {CO}_{2} ( g ; eq . 2 ) * ( 1 - ( {CO}_{2} analytical uncertainty \% / 100 ) ) \qquad ( 13 )$$

$$Sensor - Corrected Bicarbonate Generated ( g ) = ( Bicarbonate Generated ( g ; eq . 3 ) * ( 1 - ( {HCO}_{3} analytical uncertainty \% / 100 ) ) \qquad ( 14 )$$

$$Sensor - Corrected Carbonate Generated ( g ) = ( Carbonate Generated ( g ; eq . 4 ) * ( 1 - ( {CO}_{3} analytical uncertainty \% / 100 ) ) \qquad ( 15 )$$

Independent sensor deployment and modeling, performed by independent third party modeling and environmental sensing entities, provides a further validation of components assumed in the above equations. For example, speed and fractional volume of excess CO₂ outgassing from the treated water at the project location to the atmosphere are validated in the case of CDR of biogenic CO₂ in natural waters. Here, baseline DIC and pH data are combined with temperature and other environmental metrics like wind speed and slow rates to be integrated into basin-level outgassing rate estimates. While this approach assumes that excess CO₂ outgasses from high CO₂ coastal waters at a rate significantly faster than other biogenic CO₂ carbon removal and storage pathways (such as biochar), quantitative confirmation that the CO₂ outgassing rate is <1 year (i.e., the residence time of the excess CO₂ in the surface water is <1 year) will provide confidence that the project and neutralized CO₂ constitutes and immediate, additional, and climatologically significant CO₂ removal and storage that quantifiably reduces the annual atmospheric CO₂ invasion.

In addition, the spatial fate and transport of the produced HCO₃, CO₃, and alkaline elements in the specific water at deployment is validated. Here, the transport fate of the plume of treated water is assessed geospatially using known flow, bathymetric, and tidal information to project the water body mixing and transport fate, which informs the quantitative chemical assessments described below.

Furthermore, the chemical fate of the HCO₃, CO₃, and alkaline elements with respect to carbonate formation and CO₂ release are validated. Here, pre-and post-treatment DIC and pH are used in numerical box model equations to trace the chemical fate of the plume of treated water as it is transported and diluted by the surrounding un-treated waters identified above, quantifying the fraction of produced HCO3 and CO3 that could be lost by subsequent pH or alkalinity-driven transformations. Because of considerable uncertainties introduced in this exercise (from spatiotemporal variability in the surrounding un-treated water chemistry not directly measured in the project), this is a binary assessment on the credit generated, with <50% estimated loss being considered a fully permanent storage product.

Lastly, the ecological impacts from the release of HCO₃, CO₃, and alkaline elements, and the associated pH change are validated. Here, standard, low-cost water quality and biological proxy sensors (e.g., DO) and sample analyses (e.g., Chlorophyll-a) are deployed at regular intervals at the same frequency as DIC validation analyses described above, with post-treatment DO and CHI-a compared to further downstream (50 m) locations. Statistically significant variability between the two sites in these proxies should trigger a project halt, with subsequent repeated analyses to assess if the project water treatment effluent, or natural variability, is the driver of the observed change. If the former, additional down-stream sampling should proceed over the project lifespan at 50, 100, and 200 m at a distribution consistent with the plume transport identified above, with the independent third-party responsible for determining if the significant variability is indicative of a safe or unsafe project outcome.

Thus, in some examples, methods and systems described include a dissolved inorganic carbon (DIC) machine analyzing a sample of the liquid in the container to output a lab measurement of an amount of dissolved aqueous carbon in the sample independent from the autonomous measurement sensing system, and control systems independently verify the measured amount of dissolved aqueous carbon by the autonomous measurement sensing system based on comparison of the lab measurement of the amount of dissolved aqueous carbon in the sample and the measured amount of dissolved aqueous carbon.

In other examples, methods and systems described perform a potentiometric titration of a sample of the liquid (via a titrator) to output a lab measurement of alkalinity of the liquid, and control systems independently verify the measurement of alkalinity of the liquid held in the container by the autonomous measurement sensing system based on comparison of the lab measurement of alkalinity of the liquid and the measurement of alkalinity of the liquid held in the container.

### Conclusion

Carbon chemistry in waters present an ideal opportunity to capture, remove, and store a climatologically significant volume of CO₂ from the atmosphere system. Converting dissolved CO₂ to HCO₃ and CO₃ in waters through the addition of alkalinity is particularly advantageous, as it couples the removal and storage of the CO₂ in one chemical reaction, requiring no additional CO₂ transport or storage reservoirs, and can provide ecosystem co-benefits through acidification reduction. Given the highly dynamic nature of carbon in water, the deployment of this kind of carbon cycle intervention requires direct, closed system control and measurement to ensure that the negative unintended consequences on both carbon cycling and ecosystem dynamics are avoided, and to accurately determine how successful the CO₂ neutralization is at any given time.

Examples described herein include a fully-measured approach to CO₂ removal and storage in high CO₂ waters and capture and storage of gaseous CO₂ using a range of waters, and couple a real-time sensing platform for the full carbonate system including CO₂, HCO₃, CO₃, and other key water quality conditions (e.g., pH) with the accelerated dissolution of a wide range of alkaline materials in a low-energy, submerged closed system to deliver rapid, scalable, and controlled release of alkalinity to drive a measurable CO₂ neutralization which conforms to the above calculations for refined LCA and MRV. This system can leverage natural water flow for minimal pumping and low input emissions, and enables the use of variable alkaline materials to maintain low material acquisition and transport emissions at any given location.

Within examples, the systems and methods of the embodiments are embodied and/or implemented at least in part as a machine configured to receive a computer-readable medium storing computer-readable instructions. For example, the control system 110 includes a processor for executing instructions to perform functions described herein. The instructions are executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions are executed by computer-executable components integrated with apparatuses and networks of the type described above. The computer-readable medium is stored on any suitable computer readable media such as RAMs, ROMs, flash memory, EEPROMs, optical devices (CD or DVD), hard drives, floppy drives, or any suitable device. The computer-executable component is a processor, but any suitable dedicated hardware device can (alternatively or additionally) execute the instructions.

In one variation, a system comprises of one or more computer-readable mediums (e.g., non-transitory computer-readable mediums) storing instructions that, when executed by the one or more computer processors, cause a computing platform to perform operations comprising those of the system or method described herein such as: filling a management receptacle with water; measuring the water carbon concentration and/or other water conditions; and modifying the water, and dispensing the water.

The net carbon removal and storage quantifications, as described above, form a rules-based analysis of the measurements or data output from the sensors to solve technical problems associated with carbon removal and reporting requirements. Thus, outputs or measurements of sensors are transformed into quantifications of an amount of carbon removed from a particular water source.

In addition, the validation components and steps described above in Equations (1)-(15) are performed by a computing device as well.

The control systems and computing devices are useful for calculating ecological and water quality impact assessments based on real-time sensing and LCA and MRV calculations.

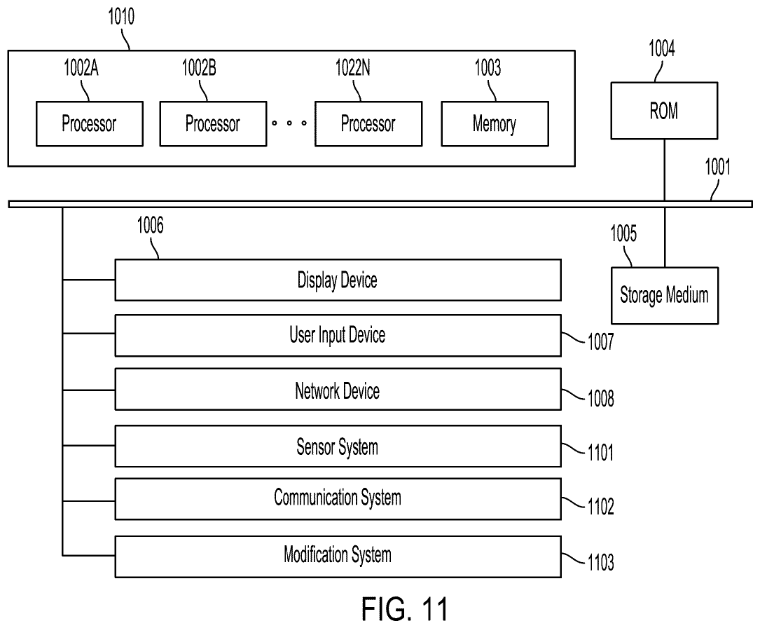

FIG. 11 is an example of a computer architecture diagram of one implementation of the control system 110. In some implementations, the control system 110 is implemented in a plurality of devices in communication over a communication channel and/or network. In some implementations, the elements of the control system 110 are implemented in separate computing devices. In some implementations, two or more of the system elements are implemented in same devices. The system and portions of the system may be integrated into a computing device or system that can serve as or within the system.

A communication channel 1001 interfaces with the processors 1002A-1002N, the memory (e.g., a random access memory (RAM)) 1003, a read only memory (ROM) 1004, a processor-readable storage medium 1005, a display device 1006, a user input device 1007, and a network device 1008. As shown, the computer infrastructure is used in connecting sensor system 1101, communication system 1102, modification system 1103, and/or other suitable computing devices.

The processors 1002A-1002N may take many forms, such CPUs (Central Processing Units), GPUs (Graphical Processing Units), microprocessors, ML/DL (Machine Learning/Deep Learning) processing units such as a Tensor Processing Unit, FPGA (Field Programmable Gate Arrays, custom processors, and/or any suitable type of processor.

The processors 1002A-1002N and the main memory 1003 (or some sub-combination) can form a processing unit 1010. In some embodiments, the processing unit includes one or more processors communicatively coupled to one or more of a RAM, ROM, and machine-readable storage medium; the one or more processors of the processing unit receive instructions stored by the one or more of a RAM, ROM, and machine-readable storage medium via a bus; and the one or more processors execute the received instructions. In some embodiments, the processing unit is an ASIC (Application-Specific Integrated Circuit). In some embodiments, the processing unit is a SoC (System-on-Chip). In some embodiments, the processing unit includes one or more of the elements of the system.

A network device 1008 provides one or more wired or wireless interfaces for exchanging data and commands between the system and/or other devices, such as devices of external systems. Such wired and wireless interfaces include, for example, a universal serial bus (USB) interface, Bluetooth interface, Wi-Fi interface, Ethernet interface, near field communication (NFC) interface, and the like.

Computer and/or machine-readable executable instructions comprising of configuration for software programs (such as an operating system, application programs, and device drivers) can be stored in the memory 1003 from the processor-readable storage medium 1005, the ROM 1004 or any other data storage system.

When executed by one or more computer processors, the respective machine-executable instructions are accessed by at least one of processors 1002A-1002N (of a processing unit 1010) via the communication channel 1001, and then executed by at least one of processors 1001A-1001N. Data, databases, data records or other stored forms data created or used by the software programs can also be stored in the memory 1003, and such data is accessed by at least one of processors 1002A-1002N during execution of the machine-executable instructions of the software programs.

The processor-readable storage medium 1005 is one of (or a combination of two or more of) a hard drive, a flash drive, a DVD, a CD, an optical disk, a floppy disk, a flash storage, a solid state drive, a ROM, an EEPROM, an electronic circuit, a semiconductor memory device, and the like. The processor-readable storage medium 1005 can include an operating system, software programs, device drivers, and/or other suitable sub-systems or software.

As used herein, first, second, third, etc. are used to characterize and distinguish various elements, components, regions, layers and/or sections. These elements, components, regions, layers and/or sections should not be limited by these terms. Use of numerical terms may be used to distinguish one element, component, region, layer and/or section from another element, component, region, layer and/or section. Use of such numerical terms does not imply a sequence or order unless clearly indicated by the context. Such numerical references may be used interchangeable without departing from the teaching of the embodiments and variations herein.

Different examples of the system(s), device(s), and method(s) disclosed herein include a variety of components, features, and functionalities. It should be understood that the various examples of the system(s), device(s), and method(s) disclosed herein may include any of the components, features, and functionalities of any of the other examples of the system(s), device(s), and method(s) disclosed herein in any combination or any sub-combination, and all of such possibilities are intended to be within the scope of the disclosure.

For the purposes of describing and defining examples herein, it is noted that terms “substantially”, “about”, or “approximately” are utilized herein to represent an inherent degree of uncertainty attributed to any quantitative comparison, value, measurement, or other representation. The terms “substantially”, “about,” and “approximately”, when utilized herein, represent the degree by which a quantitative representation may vary from a stated reference without resulting in a change in a basic function of the subject matter at issue, such as varying by 0-3% of the quantitative measurement.

As a person skilled in the art will recognize from the previous detailed description and from the figures and claims, modifications and changes can be made to the embodiments of the invention without departing from the scope of this invention as defined in the following claims.

What is claimed is:

1. A method for quantifying an amount of carbon dioxide captured from liquid, comprising:
receiving, from an autonomous measurement sensing system, information indicating a measured amount of dissolved aqueous carbon, which is generated by dosing of material into a container holding the liquid that reacts with carbon dioxide in the liquid;
receiving, from the autonomous measurement sensing system, information indicating a measured amount of carbon dioxide reduction (CDR) in the liquid held in the container, which is calculated by measurements of the liquid at an input to the container pre-treatment and the liquid at an output of the container post-treatment after dosing of the material into the container for reaction with the carbon dioxide in the liquid;
performing, by a control system including a processor executing first instructions, a first verification of a volume of carbon captured and stored as bicarbonate and carbonate in the container based on a comparison of the measured amount of dissolved aqueous carbon and the measured amount of carbon dioxide reduction (CDR);
receiving a measurement of alkalinity of the liquid held in the container after the dosing of material into the container;
based on the measurement of alkalinity of the liquid and the measured amount of dissolved aqueous carbon, generating a calculated amount of carbon dioxide reduction (CDR);
performing, by the control system including the processor executing second instructions, a second verification of the volume of carbon captured and stored as bicarbonate and carbonate based on a comparison of the measured amount of carbon dioxide reduction (CDR) and the calculated amount carbon dioxide reduction (CDR); and
based on either of the first verification or the second verification failing, providing instructions to change an amount of the dosing of the material into the container.

2. The method of claim 1, further comprising:
based on either of the first verification or the second verification failing, triggering shut-down of the dosing of the material into the container via communication with a control system configured to control the dosing.

3. The method of claim 1, further comprising:
based on either of the first verification or the second verification failing, requesting from the autonomous measurement sensing system a second set of measurements indicating an updated measured amount of dissolved aqueous carbon and an updated measured amount of carbon dioxide reduction (CDR) in the liquid held in the container.

4. The method of claim 1, wherein performing the first verification of the volume of carbon captured and stored as bicarbonate and carbonate in the container comprises:
determining whether the measured amount of dissolved aqueous carbon and the measured amount of carbon dioxide reduction (CDR) are approximately equal.

5. The method of claim 1, wherein performing the second verification of the volume of carbon captured and stored as bicarbonate and carbonate in the container comprises:
determining whether the measured amount of carbon dioxide reduction (CDR) and the calculated amount carbon dioxide reduction (CDR) are approximately equal.

6. The method of claim 1, further comprising:
quantifying an amount of volatile impurities in the liquid that impact the volume of carbon captured and stored as bicarbonate and carbonate in the container.

7. The method of claim 1, further comprising:
receiving a first initial measurement of alkalinity of the liquid held in the container prior to dosing of material into the container;
receiving a second initial measurement of alkalinity of the liquid held in the container after injection of carbon dioxide into the container and still prior to dosing of material into the container; and
based on the first initial measurement of alkalinity and the second initial measurement of alkalinity being approximately equal, quantifying an amount of volatile impurities in the liquid as nominal.

8. The method of claim 7, further comprising:
based on the second initial measurement of alkalinity being less than the first initial measurement of alkalinity by a threshold amount, determining an amount of additional dosing of the material into the container that is required to balance the alkalinity to a desired level;
determining an amount of bicarbonate and carbonate generated by the additional dosing of the material into the container; and
separately accounting for the amount of bicarbonate and carbonate generated by the additional dosing of the material into the container to balance alkalinity of the liquid and the volume of carbon captured and stored as bicarbonate and carbonate in the container based on the dosing of the material to neutralize carbon in the liquid.

9. The method of claim 1, further comprising:
a dissolved inorganic carbon (DIC) machine analyzing a sample of the liquid to output a lab measurement of an amount of dissolved aqueous carbon in the sample independent from the autonomous measurement sensing system; and
independently verifying the measured amount of dissolved aqueous carbon by the autonomous measurement sensing system based on comparison of the lab measurement of the amount of dissolved aqueous carbon in the sample and the measured amount of dissolved aqueous carbon.

10. The method of claim 1, further comprising:
performing a potentiometric titration of a sample of the liquid to output a lab measurement of alkalinity of the liquid; and
independently verifying the measurement of alkalinity of the liquid held in the container by the autonomous measurement sensing system based on comparison of the lab measurement of alkalinity of the liquid and the measurement of alkalinity of the liquid held in the container.

11. A system for quantifying an amount of carbon dioxide captured from liquid, comprising:
a container including an inlet to receive liquid and an outlet to release the liquid, the container for holding the liquid;
an autonomous measurement sensing system including one or more sensors coupled to the container to measure carbon content in the liquid that is held in the container; and
a control system including a processor for executing instructions to perform functions of:
receiving, from the autonomous measurement sensing system, information indicating a measured amount of dissolved aqueous carbon, which is generated by dosing of material into the container holding the liquid that reacts with carbon dioxide in the liquid;
receiving, from the autonomous measurement sensing system, information indicating a measured amount of carbon dioxide reduction (CDR) in the liquid held in the container, which is calculated by measurements of the liquid at an input to the container pre-treatment and the liquid at an output of the container post-treatment after dosing of the material into the container for reaction with the carbon dioxide in the liquid;
performing a first verification of a volume of carbon captured and stored as bicarbonate and carbonate in the container based on a comparison of the measured amount of dissolved aqueous carbon and the measured amount of carbon dioxide reduction (CDR);
receiving a measurement of alkalinity of the liquid held in the container after the dosing of material into the container;
based on the measurement of alkalinity of the liquid and the measured amount of dissolved aqueous carbon, generating a calculated amount of carbon dioxide reduction (CDR);
performing a second verification of the volume of carbon captured and stored as bicarbonate and carbonate based on a comparison of the measured amount of carbon dioxide reduction (CDR) and the calculated amount carbon dioxide reduction (CDR); and
based on either of the first verification or the second verification failing, providing instructions to change an amount of the dosing of the material into the container.

12. The system of claim 11, wherein the control system further executes the instructions to perform functions of:
based on either of the first verification or the second verification failing, trigger shut-down of the dosing of the material into the container.

13. The system of claim 11, wherein the control system further executes the instructions to perform functions of:
based on either of the first verification or the second verification failing, request from the autonomous measurement sensing system a second set of measurements indicating an updated measured amount of dissolved aqueous carbon and an updated measured amount of carbon dioxide reduction (CDR) in the liquid held in the container.

14. The system of claim 11, wherein the function of performing the first verification of the volume of carbon captured and stored as bicarbonate and carbonate in the container comprises:
determining whether the measured amount of dissolved aqueous carbon and the measured amount of carbon dioxide reduction (CDR) are approximately equal.

15. The system of claim 1, wherein the function of performing the second verification of the volume of carbon captured and stored as bicarbonate and carbonate in the container comprises:
determining whether the measured amount of carbon dioxide reduction (CDR) and the calculated amount carbon dioxide reduction (CDR) are approximately equal.

16. The system of claim 1, wherein the control system further executes the instructions to perform functions of:
quantifying an amount of volatile impurities in the liquid that impact the volume of carbon captured and stored as bicarbonate and carbonate in the container.

17. The system of claim 1, wherein the control system further executes the instructions to perform functions of:
receiving a first initial measurement of alkalinity of the liquid held in the container prior to dosing of material into the container;
receiving a second initial measurement of alkalinity of the liquid held in the container after injection of carbon dioxide into the container and still prior to dosing of material into the container; and
based on the first initial measurement of alkalinity and the second initial measurement of alkalinity being approximately equal, quantifying an amount of volatile impurities in the liquid as nominal.

18. The method of claim 17, wherein the control system further executes the instructions to perform functions of:
based on the second initial measurement of alkalinity being less than the first initial measurement of alkalinity by a threshold amount, determining an amount of additional dosing of the material into the container that is required to balance the alkalinity to a desired level;
determining an amount of bicarbonate and carbonate generated by the additional dosing of the material into the container; and
separately accounting for the amount of bicarbonate and carbonate generated by the additional dosing of the material into the container to balance alkalinity of the liquid and the volume of carbon captured and stored as bicarbonate and carbonate in the container based on the dosing of the material to neutralize carbon in the liquid.

19. The system of claim 11, further comprising:
a dissolved inorganic carbon (DIC) machine analyzing a sample of the liquid to output a lab measurement of an amount of dissolved aqueous carbon in the sample independent from the autonomous measurement sensing system; and
wherein the control system further executes the instructions to perform functions of:
independently verifying the measured amount of dissolved aqueous carbon by the autonomous measurement sensing system based on comparison of the lab measurement of the amount of dissolved aqueous carbon in the sample and the measured amount of dissolved aqueous carbon.

20. The system of claim 11, further comprising:
a titrator performing a potentiometric titration of a sample of the liquid to output a lab measurement of alkalinity of the liquid; and
wherein the control system further executes the instructions to perform functions of:
independently verifying the measurement of alkalinity of the liquid held in the container by the autonomous measurement sensing system based on comparison of the lab measurement of alkalinity of the liquid and the measurement of alkalinity of the liquid held in the container.
